(19) United States
(12) Patent Application Publication
(10) Pub. No.: US 2026/0321411 A1
(43) Pub. Date: Oct. 15, 2026

(54) HAND-HELD MULTIMODAL PREDICATE DIAGNOSTIC SYSTEM FOR STROKE DETECTION USING MOBILE DEVICE SENSORS

(71) Applicant: George William Bickerstaff, III, Greenwich, CT (US)

(72) Inventor: George William Bickerstaff, III, Greenwich, CT (US)

(21) Appl. No.: 19/466,792

(22) Filed: Feb. 2, 2026

(51) Int. Cl. G16H 40/63; G06F 8/65; H04L 9/06

(52) U.S. Cl. CPC G16H 40/63; G06F 8/65; H04L 9/0631; H04L 9/0643

(57) ABSTRACT

A multimodal predicate diagnostic system for stroke detection utilizes secured mobile devices to monitor users in consumer environments. The system aggregates motion data using optimized sampling and cryptographic validation, applies a hybrid convolutional neural-network and decision-tree model optimized through transfer learning and data augmentation against an adaptive gait baseline, computes risk confidence scores in real time, and generates encrypted alerts through network integration.

### STATEMENT REGARDING FEDERALLY SPONSORED RESEARCH OR DEVELOPMENT

Not applicable.

### INCORPORATION BY REFERENCE

None.

### FIELD OF THE INVENTION

The present invention relates to mobile health monitoring systems, cryptographic diagnostic architectures, and artificial-intelligence-driven analytics for consumer and clinical environments across a range of physiological conditions. More particularly, the invention encompasses hand-held, mobile device-based predicate diagnostic frameworks that analyze multimodal sensor data using adaptive baselines for real-time detection of neurological events including stroke and transient ischemic attack (TIA). The invention further incorporates transfer learning from public datasets, artificial-intelligence governance layers for regulated compliance, and extensible embodiments applicable to cardiovascular irregularities, mobility disorders, and additional physiological monitoring scenarios. The invention addresses the integration of secure artificial intelligence in portable diagnostics to facilitate personal health management, telemedicine delivery, and preventive care systems while preserving data confidentiality, integrity, and auditability.

### BACKGROUND OF THE INVENTION

Stroke and transient ischemic attacks represent substantial public-health challenges requiring rapid identification to reduce irreversible neurological damage and long-term disability. Existing consumer technologies often fail to provide accessible and continuous monitoring suitable for dynamic daily life and frequently do not capture subtle early indicators such as gait asymmetry, balance deviation, or stride irregularity. Conventional approaches rely upon sporadic clinical evaluation or fixed diagnostic equipment that are not practical for ongoing personal monitoring outside institutional environments.

Prior art systems, including fall-detection wearables relying primarily on threshold-based accelerometer triggers, mobile gait analytics platforms oriented toward general fitness tracking, and generic machine-learning anomaly detection systems lacking cryptographic validation and predicate-specific logic, do not adequately address secure real-time neurological risk detection. Such systems typically emphasize single-sensor heuristics, lack adaptive baseline personalization, and do not provide tamper-resistant computational environments or formal predicate rule frameworks. Accordingly, a technical need exists for a hand-held multimodal diagnostic system capable of continuous motion analysis, secure on-device artificial intelligence processing, adaptive baseline modeling, and low-latency alerting while maintaining strict privacy and data integrity controls across heterogeneous usage conditions.

### SUMMARY OF THE INVENTION

The invention provides a predicate diagnostic system for detecting stroke and transient ischemic attacks using hand-held mobile devices. The term “predicate diagnostic” refers to a rule-based analytical framework that evaluates conditional physiological criteria to classify health events by integrating multimodal sensor data with adaptive computational models and defined confidence thresholds. The system comprises a sensor data aggregator optimized for mobile hardware, a predicate analysis engine implementing adaptive gait baselines, a stroke risk classifier configured for sub-second confidence computation, and an alert module configured for secure transmission and prioritized notification routing. Through utilization of mobile connectivity protocols, hardware-level encryption, and artificial-intelligence architectures optimized via transfer learning and generative data augmentation, the system identifies physiological deviations while maintaining confidentiality, integrity, and verifiable audit trails.

Technical advantages of the invention include reduction of false positives through adaptive filtering and quorum-based signal validation, on-device privacy preservation through hardware-anchored encryption and trusted execution boundaries, sub-second latency for risk computation even under high processing load, and independence from external infrastructure for continuous monitoring. Additional advantages include scalability across device classes, interoperability with standardized medical data exchange formats, and extensibility to additional physiological monitoring domains without architectural redesign.

### BRIEF DESCRIPTION OF THE DRAWINGS

FIG. 1—SYSTEM ARCHITECTURE

FIG. 1A—SENSOR DATA AGGREGATOR CONFIGURATION

This subfigure illustrates the configuration for collecting multimodal data from mobile device sensors including accelerometers, gyroscopes, magnetometers, and auxiliary biometric inputs. The diagram depicts normalization of sampling rates, buffering of high-fidelity streams, and battery-aware scheduling policies to maintain continuous operation without excessive power consumption. Secure interfaces to device application programming interfaces and permission controls are shown to ensure controlled data access and integrity throughout aggregation.

FIG. 1B—PREDICATE ANALYSIS ENGINE FLOW

This subfigure depicts the logical workflow of the predicate analysis engine as it receives preprocessed sensor inputs and applies conditional rules alongside learned feature vectors. Decision branches illustrate anomaly detection paths, threshold evaluations, and escalation logic used to differentiate normative gait behavior from acute neurological deviations. Governance checkpoints and audit hooks are shown to demonstrate traceability, reproducibility, and explainability of analytical outcomes.

FIG. 1C—STROKE RISK CLASSIFIER ALGORITHM

This subfigure presents the algorithmic structure for computing risk confidence scores using hybrid model outputs and adaptive baseline comparisons. Interactions between temporal feature windows, historical baselines, and confidence calibration modules are illustrated to show validated score production within constrained latency targets. Embedded security layers and on-device execution boundaries emphasize confidentiality and tamper resistance.

FIG. 1D—ALERT MODULE INTEGRATION

This subfigure illustrates integration of the alert module with mobile networking stacks, notification services, and emergency contact routing mechanisms. Priority queues, retry policies, and encryption channels are depicted to ensure timely and reliable delivery under variable connectivity conditions. User acknowledgment pathways and escalation rules are shown to support rapid response while maintaining verifiable delivery records.

FIG. 1E—DEVICE SYNCHRONIZATION PROTOCOL

This subfigure describes the protocol used to synchronize session data across device restarts, software updates, and intermittent connectivity states. Secure handshakes, timestamp reconciliation, and conflict-resolution rules maintain continuity and prevent duplication or data loss. Cryptographic verification steps ensure integrity and provenance across sessions.

FIG. 2—DATA PROCESSING WORKFLOW

FIG. 2A—MOTION DATA COLLECTION PIPELINE

This subfigure outlines the end-to-end pipeline for motion data acquisition including trigger thresholds, buffering strategies, and adaptive sampling intervals. Noise-reduction filters, calibration routines, and digital signing steps prepare raw signals for downstream analytics. Quality-assurance checkpoints enforce minimum signal-to-noise ratios and discard corrupted frames.

FIG. 2B—PREDICATE RULE APPLICATION LOGIC

This subfigure details logical structures used to evaluate conditional predicates against normalized sensor features and adaptive baselines. Variable weighting, confidence bands, and rule-override safeguards handle diverse movement patterns without exceeding diagnostic boundaries. Logging and explainability modules provide human-readable justifications for outcomes.

FIG. 2C—RISK SCORING COMPUTATION

This subfigure illustrates composite risk-score computation by integrating current feature vectors with historical trend data and calibration curves. Formatting layers and serialization routines prepare outputs for secure storage, display, and transmission. Performance optimizations achieve rapid execution on mobile processors while maintaining numerical stability.

FIG. 2D—ALERT GENERATION SEQUENCE

This subfigure shows ordered sequences for alert creation, encryption, routing, and confirmation tracking following threshold breaches. Event logs, escalation timers, and redundancy channels ensure delivery resilience and accountability. Safety-prioritization mechanisms minimize latency while preventing duplicate notifications.

FIG. 2E—DATA VALIDATION CHECKPOINT This subfigure depicts validation steps comparing live signals against adaptive baselines and cryptographic signatures to verify authenticity and integrity. Error-handling branches and quarantine buffers isolate anomalous or suspicious data segments. Compliance flags and audit trails support regulatory documentation and post-event review.

FIG. 3—USER INTERFACE DESIGN

FIG. 3A—REAL-TIME MONITORING DISPLAY LAYOUT

This subfigure presents a dashboard layout aggregating live metrics, trend lines, and status indicators onto a unified screen. Layered overlays and configurable widgets enable simultaneous monitoring of motion, baseline deviation, and alert readiness. Accessibility features and contrast safeguards enhance usability across diverse populations.

FIG. 3B—ALERT NOTIFICATION PANEL DESIGN

This subfigure illustrates a notification panel displaying priority levels, confidence scores, and recommended actions. Interactive controls for acknowledgment, escalation, and contact selection support rapid decision-making. Visual and haptic feedback mechanisms ensure awareness under varied conditions.

FIG. 3C—HISTORICAL DATA VISUALIZATION

This subfigure shows longitudinal charts and comparative timelines enabling review of gait metrics and risk scores over selectable intervals. Zoom, filter, and annotation tools facilitate detailed examination of specific data points. Export and sharing options support clinical consultations and recordkeeping.

FIG. 3D—USER SETTINGS INTERFACE This subfigure depicts configuration panels allowing authorized users to adjust thresholds, notification preferences, and privacy controls. Role-based access restrictions and confirmation dialogs prevent unauthorized or accidental changes. Preset profiles and reset options streamline personalization while maintaining safety defaults.

FIG. 3E—EMERGENCY CONTACT CONFIGURATION

This subfigure describes setup screens linking trusted contacts and emergency services. Verification workflows, encryption indicators, and consent prompts protect privacy and ensure valid routing information. Update and audit features maintain accuracy over time.

FIG. 4—HARDWARE INTEGRATION SCHEMATIC

FIG. 4A—SENSOR ARRAY LAYOUT

This subfigure illustrates physical placement and logical grouping of motion and biometric sensors relative to the processing unit. Shielding elements, isolation traces, and secure buses reduce interference and unauthorized access. Form-factor considerations optimize portability and ergonomic handling.

FIG. 4B—CONNECTIVITY PROTOCOLS

This subfigure shows wireless and wired communication pathways including secure pairing, handshake verification, and session management. Continuous-monitoring channels and fallback links maintain coverage during signal fluctuations. Encryption layers and key-exchange routines preserve confidentiality and integrity.

FIG. 4C—POWER MANAGEMENT CIRCUIT

This subfigure depicts charging circuits, voltage regulators, and battery-health monitors sustaining continuous operation. Dynamic scaling and sleep-mode transitions balance performance with energy efficiency. Protective components and fail-safes mitigate overcurrent and overheating.

FIG. 4D—DATA ENCRYPTION MODULE

This subfigure illustrates a dedicated encryption module employing standardized algorithms and secure key storage. Isolated execution paths and hardware-backed keystores prevent unauthorized extraction. Integrity-checking and tamper-evident features detect intrusion attempts.

FIG. 4E—BATTERY OPTIMIZATION FLOW This subfigure describes control flows adjusting sampling frequency, processing intensity, and network usage based on battery state. Predictive models and notifications extend operational time without compromising safety thresholds. Logging and analytics refine optimization policies.

FIG. 5—VALIDATION PROCESS

FIG. 5A—DATA COLLECTION

This subfigure outlines controlled collection procedures including consent capture, anonymization, and secure storage. Sampling diversity and demographic balancing improve representativeness. Chain-of-custody records maintain provenance.

FIG. 5B—PROTOCOL

This subfigure depicts standardized validation protocols measuring sensitivity and specificity against verified outcomes. Milestone gates ensure statistical rigor. Documentation artifacts support regulatory submission.

FIG. 5C—ACCURACY EVALUATION

This subfigure shows computation of precision, recall, and area-under-curve metrics across stratified cohorts. Error analysis charts identify biases. Iterative refinement loops guide improvements.

FIG. 5D—FEEDBACK LOOP IMPLEMENTATION

This subfigure illustrates incorporation of verified feedback into adaptive model updates. Integrity checks and rollback options prevent drift. Secure deployment channels distribute validated updates.

FIG. 5E—COMPLIANCE CHECK

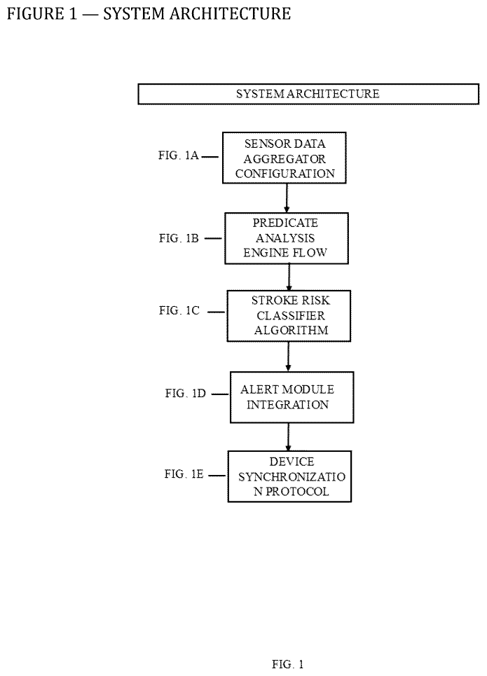

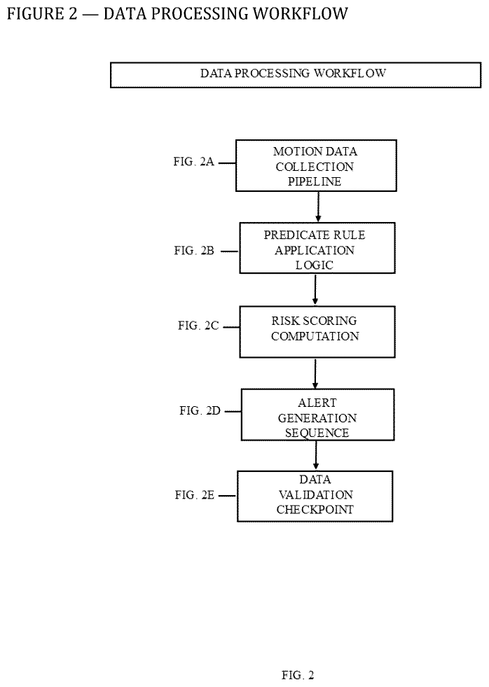

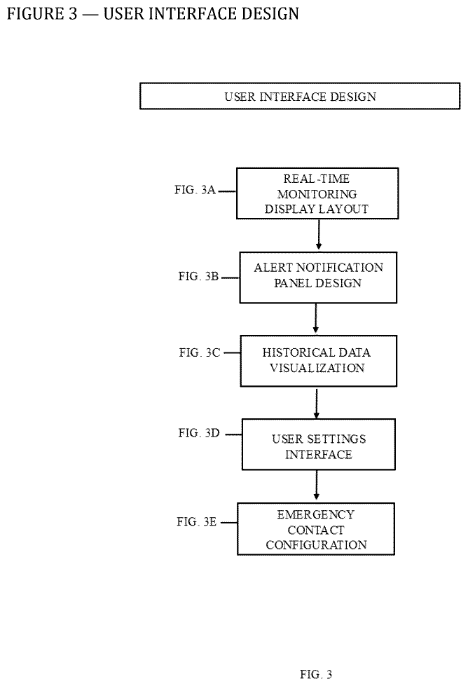

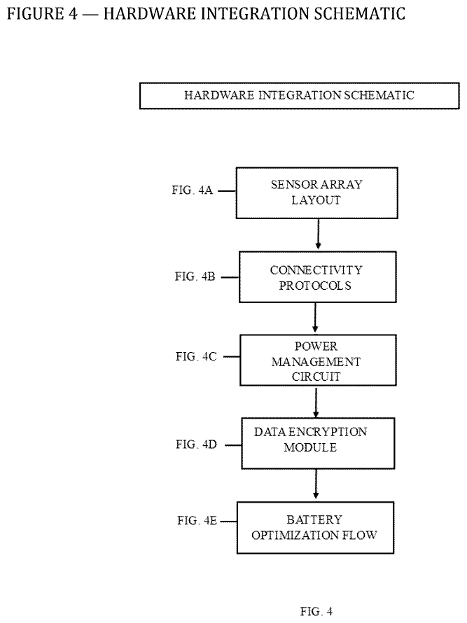

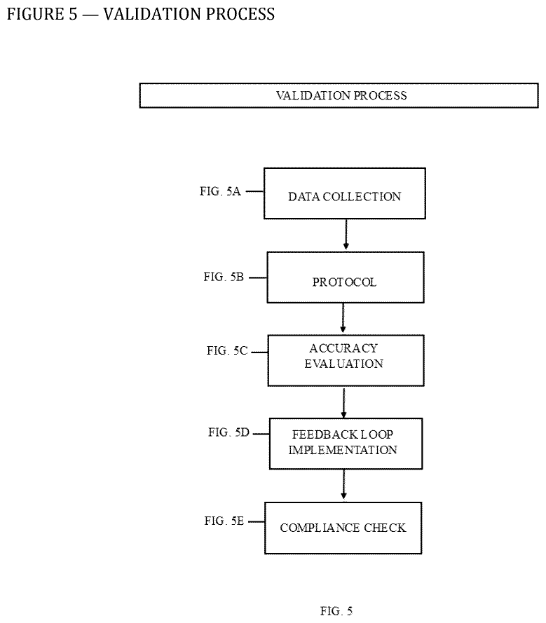

This subfigure describes compliance verification including security testing, efficacy validation, and documentation generation. Regulatory mapping tables align system behavior with standards. Continuous-monitoring hooks maintain compliance posture.

### DETAILED DESCRIPTION OF THE INVENTION

The predicate diagnostic system operates on secured mobile devices employing hardware-enforced confidentiality and integrity controls. The adaptive gait baseline is quantified through biomechanical parameters including step-length variability, swing-phase duration, and asymmetry indices normalized to defined scales. Baselines update through rolling averages of recent normative walking sessions to distinguish chronic patterns from acute neurological deviations.

Artificial-intelligence training utilizes a hybrid convolutional neural network and decision-tree architecture. Transfer learning initializes weights from public datasets while generative augmentation expands rare-event samples. Binary cross-entropy loss and stochastic gradient descent refine classification accuracy.

A Trusted Execution Environment provides hardware isolation through memory enclaves and remote attestation verified by a root of trust. Sensor fusion employs extended Kalman filtering to differentiate environmental disturbances from medically relevant gait events. Variational autoencoder architectures support dataset expansion while preserving statistical distributions.

Failure-mode safeguards address sensor drift through calibration thresholds, device misplacement through inactivity detection, low-battery operation through adaptive sampling, network loss through local storage, and conflicting signals through quorum voting. Interoperability is supported through HL7/FHIR-compatible data exchange and Bluetooth Low Energy health profiles. Scalability ranges support high-volume daily sessions while maintaining sub-second latency.

### DEFINITIONS

ADAPTIVE GAIT BASELINE—A dynamic mathematical model that adjusts to an individual's walking patterns over time to establish a personalized normative reference. It continuously updates using recent validated motion data to distinguish chronic characteristics from acute neurological deviations.
CRYPTOGRAPHIC SOFTWARE—A software module employing standardized encryption and hashing algorithms such as AES-256 and SHA-3 to protect data confidentiality and integrity. It manages key generation, storage, and verification processes to prevent unauthorized access or tampering.
DATA ENCRYPTION MODULE—A hardware or firmware component dedicated to securing multimodal sensor data through cryptographic operations. It operates in isolation from general application processes to reduce exposure to external threats and unauthorized extraction.
ENVIRONMENTAL DATA FILTER—An algorithmic mechanism that analyzes contextual motion signals to suppress false alerts caused by non-medical environmental factors. It differentiates between surface changes, device drops, and genuine physiological anomalies using sensor fusion and threshold logic.
GENERATIVE DATA AUGMENTATION—A computational process that creates synthetic training samples to expand limited diagnostic datasets. It preserves statistical distributions and feature relationships to improve model robustness and rare-event detection accuracy.
CONNECTIVITY PROTOCOL—A structured communication framework enabling secure data exchange between devices, networks, and external systems. It governs authentication, session management, and encryption standards to maintain continuity and confidentiality of transmitted information.
STROKE INDICATOR—A measurable physiological or motion-based metric that correlates with potential neurological impairment. It may include asymmetry ratios, balance deviations, or abrupt changes in gait characteristics detected through sensor analysis.
MOBILE SENSOR ARRAY—An integrated combination of motion and biometric sensors embedded within or connected to a mobile device for continuous monitoring. The array operates in coordinated fashion to collect synchronized multimodal data streams for analytical processing.
PREDICATE RULE—A conditional logical statement or set of criteria used to evaluate sensor data against predefined or learned thresholds. It forms part of the diagnostic decision framework that determines whether an alert condition is satisfied.
REAL-TIME ALERT THRESHOLD—A predefined quantitative or probabilistic boundary that triggers an immediate notification when exceeded. It is dynamically adjustable based on adaptive baselines and contextual factors to balance sensitivity and specificity.
TRUSTED EXECUTION BOUNDARY—A hardware-enforced isolation zone within a computing device that restricts access to sensitive computations and data. It ensures that critical diagnostic operations execute in a tamper-resistant environment separate from general operating system processes.

1. A computer-implemented system for predicate diagnostic detection of stroke and transient ischemic attacks using a hand-held mobile device, the system comprising:
a sensor data aggregator configured to collect multimodal motion and biometric data from at least one mobile device sensor including an accelerometer and a gyroscope;
a predicate analysis engine configured to apply conditional logical rules to normalized sensor data;
an adaptive gait baseline module configured to generate and update individualized reference metrics using rolling averages of validated walking sessions;
a stroke risk classifier configured to compute a risk confidence score based on comparisons between live sensor inputs and the adaptive gait baseline; and
an encrypted alert module configured to generate and transmit notifications through secure communication channels;
wherein the sensor data aggregator, predicate analysis engine, adaptive gait baseline module, stroke risk classifier, and encrypted alert module operate within a trusted execution environment providing hardware-enforced isolation, and
wherein the system is further configured to compute validated risk confidence scores in less than one second following receipt of sensor input data.

2. A computer-implemented method for predicate diagnostic detection of stroke and transient ischemic attacks using a hand-held mobile device, the method comprising:
collecting multimodal motion data from at least one mobile device sensor; preprocessing the collected data using normalization and noise-reduction filters; applying predicate rules to the preprocessed data using an adaptive gait baseline model; computing a risk confidence score using a machine-learning classifier; andgenerating an encrypted alert containing routing information for transmission to at least one designated recipient; wherein the method is executed within a trusted execution environment and produces a validated risk confidence score within a predefined latency threshold.

3. A non-transitory computer-readable medium storing instructions that, when executed by at least one processor of a mobile device, cause the processor to perform the method of claim 2.

4. The system of claim 1, wherein the stroke risk classifier utilizes transfer learning initialized from publicly available medical or motion datasets.

5. The system of claim 1, wherein the adaptive gait baseline module incorporates generative data augmentation to expand rare-event training samples while preserving statistical distributions.

6. The system of claim 1, wherein the encrypted alert module employs cryptographic validation including at least one of AES-256 encryption or SHA-3 hashing.

7. The system of claim 1, further comprising an environmental data filter configured to suppress false alerts caused by non-physiological disturbances including device drops or uneven walking surfaces.

8. The system of claim 1, further comprising a device synchronization protocol configured to maintain data continuity across device restarts and software updates using secure timestamp reconciliation.

9. The system of claim 1, wherein the predicate analysis engine applies quorum-based signal validation across multiple sensors before generating an alert condition.

10. The system of claim 1, wherein the trusted execution environment comprises a hardware-backed secure enclave or equivalent isolated processing region.

11. The method of claim 2, further comprising performing extended Kalman filtering to fuse motion signals and reduce environmental noise.

12. The method of claim 2, further comprising dynamically adjusting sampling frequency based on battery level and device activity state.

13. The method of claim 2, wherein generating the encrypted alert includes prioritizing emergency contacts according to predefined escalation rules.

14. The method of claim 2, further comprising logging audit trails including timestamped cryptographic signatures for regulatory documentation.

15. The non-transitory computer-readable medium of claim 3, wherein the stored instructions further cause the processor to execute a convolutional neural network in combination with a decision-tree model.

16. The non-transitory computer-readable medium of claim 3, wherein the stored instructions further cause the processor to perform adaptive baseline refinement using rolling historical data windows.

17. The system of claim 1, wherein interoperability is achieved through compliance with HL7 or FHIR medical data exchange standards.

18. The method of claim 2, further comprising isolating anomalous data segments in a quarantine buffer prior to classification.

19. The system of claim 1, wherein the encrypted alert module supports multi-channel routing including cellular, Wi-Fi, and Bluetooth Low Energy communication pathways.
